(19) United States
(12) Patent Application Publication
(10) Pub. No.: US 2026/0272705 A1
(43) Pub. Date: Sep. 17, 2026

(54) Effective Heating and Healing On Bone and Soft Tissue

(71) Applicant: John Salvo, Haddonfield, NJ (US)

(72) Inventor: John Salvo, Haddonfield, NJ (US)

(21) Appl. No.: 19/075,962

(22) Filed: Mar. 11, 2025

(51) Int. Cl. A61F 7/00

(52) U.S. Cl. CPC A61F 7/007; A61F 2007/0074; A61F 2007/008

(57) ABSTRACT

A bone osteogenesis assembly includes a wearable backpack assembly. The wearable backpack assembly includes a bag portion. The bag portion includes a pair of shoulder straps. The bag portion further having a battery housing positioned on a rear of the bag portion. The bag portion further having a circuit board housing positioned adjacent the battery housing. The bone osteogenesis further includes a sleeve component adjacent the backpack assembly. The sleeve component having a heater housing. The bone osteogenesis assembly further includes an electrical sub assembly having a battery positioned within the battery housing. The electrical sub assembly further includes a control system device positioned on a circuit board within the circuit board housing. The electrical sub assembly further includes a heater housed within the heater housing, the heater coupled to the control system device and the battery.

### FIELD OF THE INVENTION

The present invention pertains to bone healing and, more particularly, to a bone osteogenesis assembly.

### BACKGROUND

There are around 6.8 million bone fractures per year in the United States alone, which does not account for the number of fractures not treated by doctors. While fractures are very common, 95% of bone fractures require a healing time of 6-8 weeks. This healing time includes a period of immobilization where the bone is structurally set and a period of rehabilitation in which the patient regains strength lost while immobilized. This period of healing post-fracture limits a person's ability to work, play, or participate in their normal daily activities.

Recently, research has been conducted to discover ways to expedite the healing process. Several methods such as electricity, pulsed electromagnetic fields, low-intensity pulsed ultrasound, coupled capacitance stimulation, and extracorporeal shock waves have been explored as methods to accelerate osteogenesis, or bone growth, but proved to be either too costly or too invasive. One method that has the possibility of being effective at lower costs both financially and physically, however, is the application of heat to the site of the fracture.

Heat, when applied to an area of the body, causes blood vessels to dilate, improving circulation to that area. Circulation is vital to bone osteogenesis because the blood that circulates allows essential nutrients as well as oxygen to the fractured bone site. These nutrients are used to build up and re-strengthen the previously fractured bone. Improving circulation by adding heat, therefore, could be beneficial for bone repair.

Currently, there are few research studies demonstrating the effect of continuous heating on improved fracture healing times. This is due to a lack of technology needed to facilitate such bone heating for osteogenesis research. To prove the effect of continuous heating on fracture healing, a heating device that can be applied for an extended period over a bone fracture and whose temperature can be adjusted and monitored to carry out sufficient research to support the hypothesis that heating can stimulate osteogenesis must be created.

During testing, this invention will be able to sit on the fracture site of a test subject while simultaneously heating the skin and measuring its temperature The device will operate for 12 hours without inhibiting daily activities This design may be accommodated for testing subjects such as a rat with an external fixation device attached to each of its two broken humerus bones. This device is placed around one of 2 broken limbs of the rat while the other broken limb of the rat serves as a control without the use of a heating device. The testing aims to test the effectiveness of heating on the process of osteogenesis. The invention may be implemented into research practice where heating and temperature sensing will occur on a live rat. Eventually, once preliminary testing is complete, research will later expand to larger test subjects including dogs and humans. The invention may also be utilized for soft tissue healing for muscles, tendons and ligaments. The proposed design may be modified in scale to accommodate larger animals or humans. Similarly, the devices used within the proposed design will also be modified to scale in order to accommodate the larger animal or human. Ultimately, the dimension of the proposed design with the devices of the proposed design will correspond to the size of the subject or user.

### SUMMARY

A bone osteogenesis assembly includes a wearable backpack assembly. The wearable backpack assembly includes a bag portion. The bag portion includes a pair of shoulder straps. The bag portion further having a battery housing positioned on a rear of the bag portion. The bag portion further having a circuit board housing positioned adjacent the battery housing. The bone osteogenesis further includes a sleeve component adjacent the backpack assembly. The sleeve component having a heater housing. The bone osteogenesis assembly further includes an electrical sub assembly having a battery positioned within the battery housing. The electrical sub assembly further includes a control system device positioned on a circuit board within the circuit board housing. The electrical sub assembly further includes a heater housed within the heater housing, the heater coupled to the control system device and the battery.

### BRIEF DESCRIPTION OF DRAWINGS

In the following, the present invention is described in more detail with references to the drawings in which:

FIG. 1 illustrates a front, top, right side perspective view of an exemplary embodiment of the invention;

FIG. 2 illustrates a front, top, left side perspective view of another exemplary embodiment of the invention;

FIG. 3 illustrates a front, top, right side perspective view of another exemplary embodiment of the invention;

FIG. 4 illustrates a top perspective view of the invention;

FIG. 5 illustrates a top perspective view of the invention;

FIG. 6 illustrates a front perspective view of a portion of the invention;

FIG. 7 illustrates a front perspective view of a portion of the invention;

FIG. 8 illustrates a top perspective view of a portion of the invention;

FIG. 9 illustrates a top perspective view of a portion of the invention; and

FIG. 10 illustrates a front, top, right side perspective view of the invention.

### DETAILED DESCRIPTION OF THE EMBODIMENT(S)

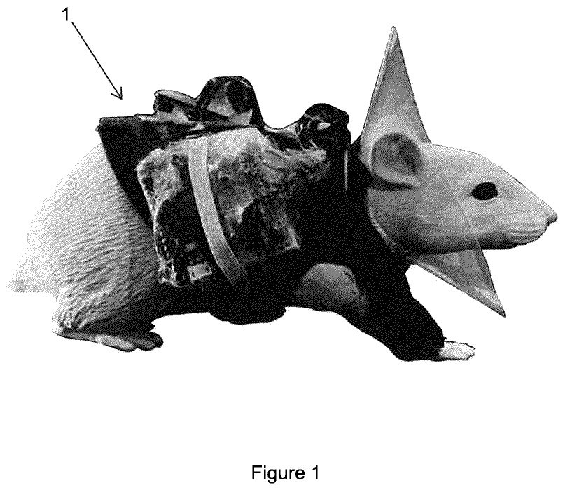

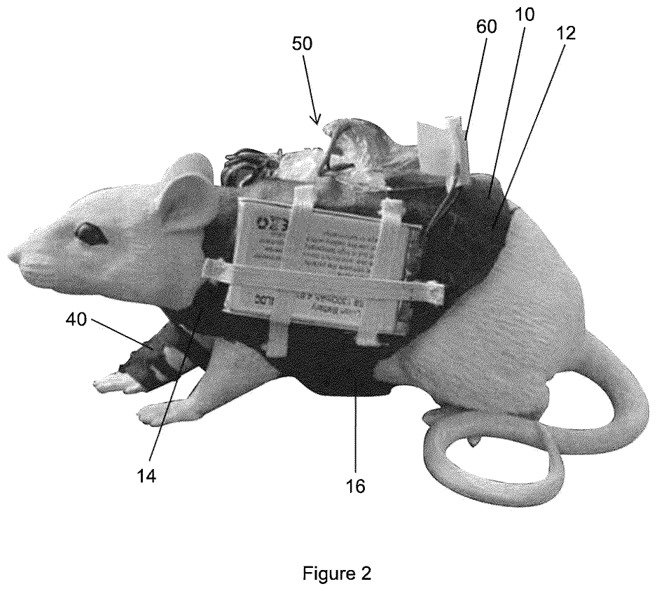

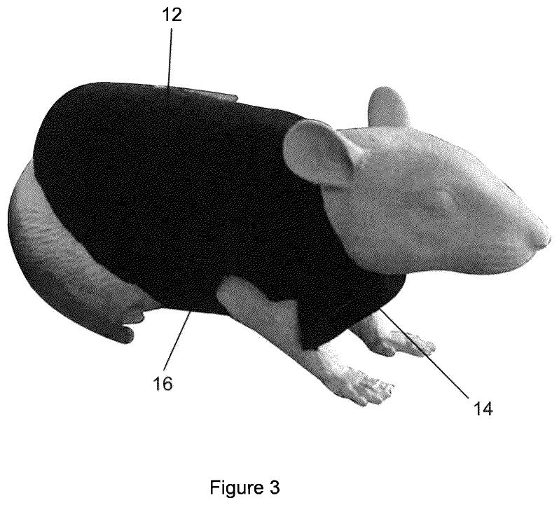

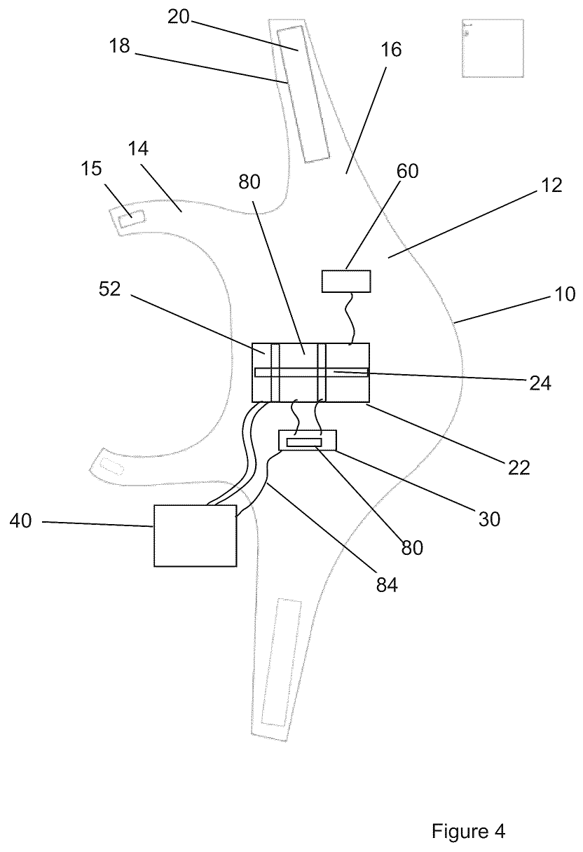

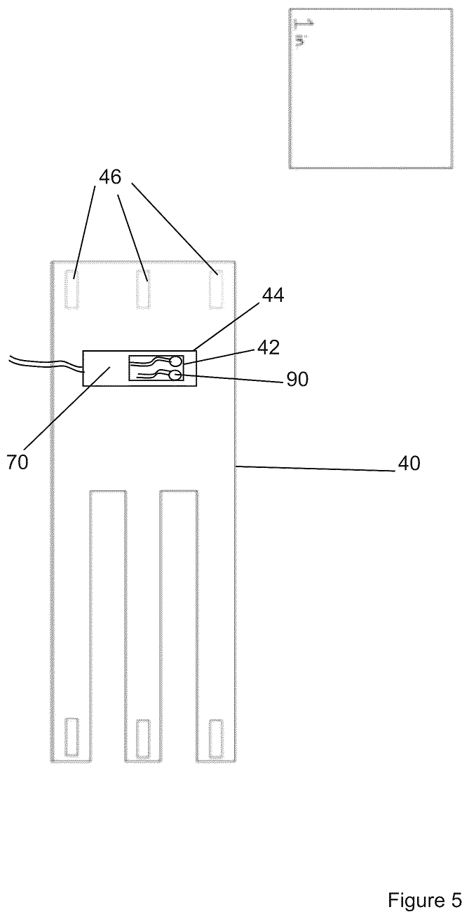

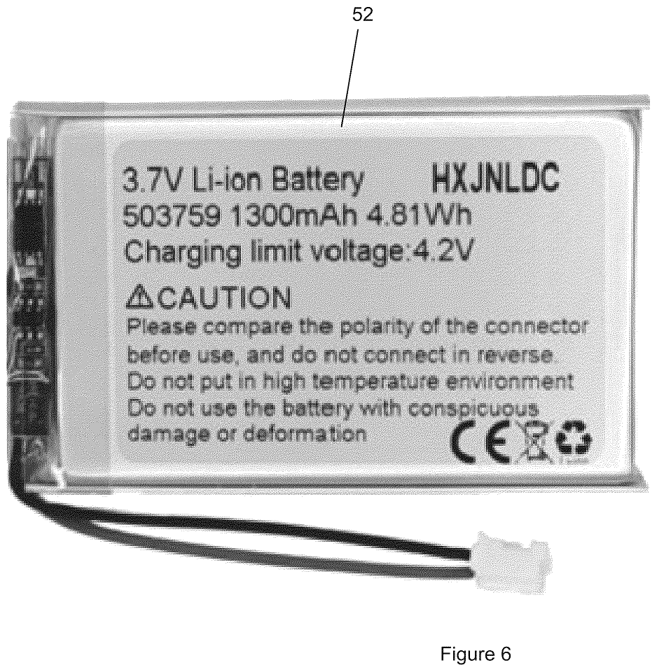

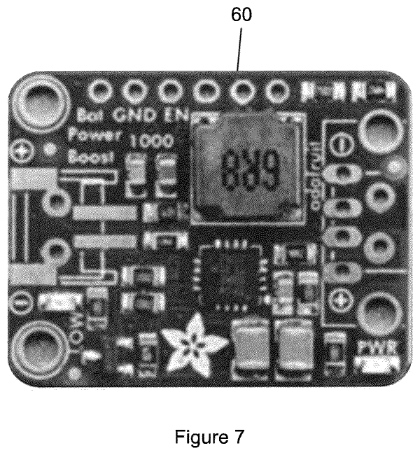

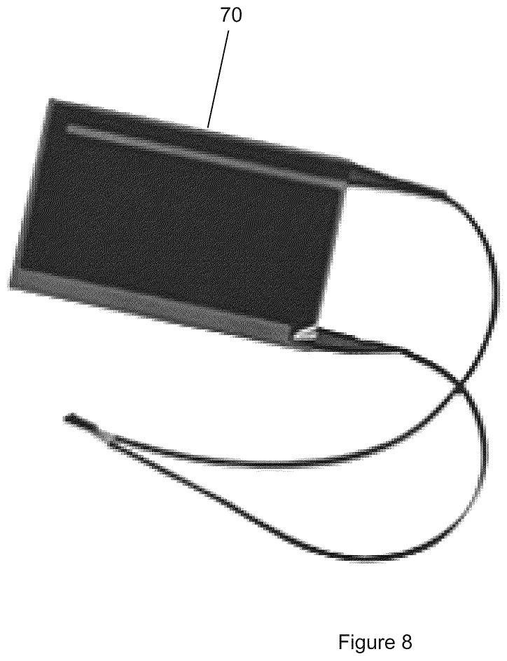

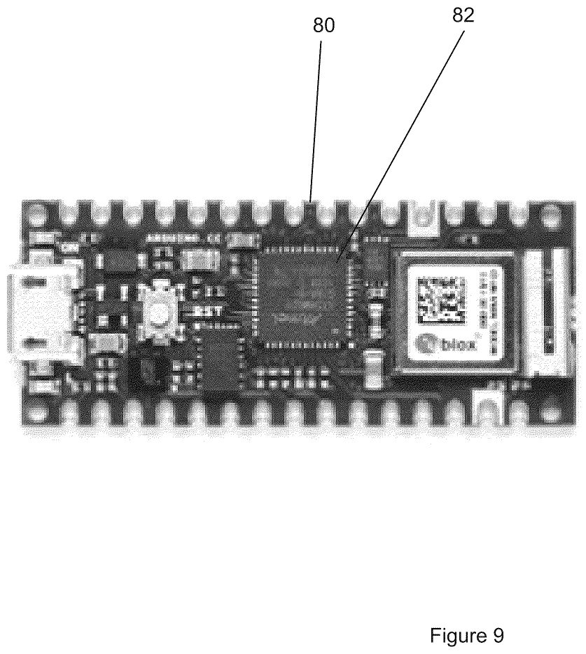

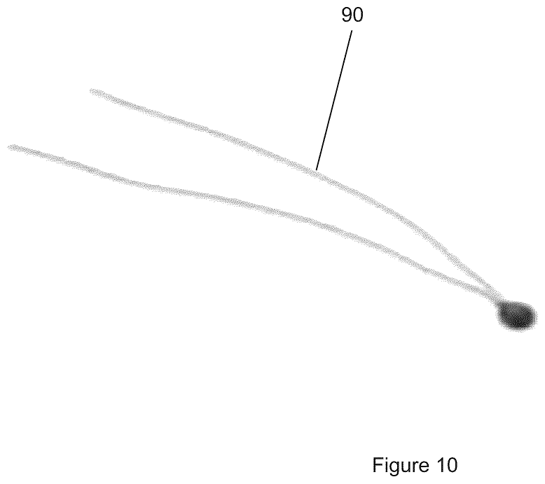

With reference to FIGS. 1-10, a bone osteogenesis assembly 1 according to the invention generally includes a wearable backpack assembly 10 having a sleeve component 40 and an electrical subassembly 50.

In an exemplary embodiment of the invention, the wearable backpack assembly 10 includes a bag portion 12. The bag portion 12 may be composed of polyester rayon spandex fabric mixture. One skilled in the art would understand the applicant's design is not the exclusive embodiment. The bag portion 12 provides a pair of shoulder straps 14. The pair of shoulder straps 14 support the bag portion 12 of the backpack assembly 10 on the back or posterior side of the bearer. The pair of shoulder straps 14 further includes a fastener 15. The fastener 15 may be a hook and loop fastener. One skilled in the art would understand the applicant's design is not the exclusive embodiment.

In an exemplary embodiment of the invention, the bag portion 12 further provides a waist belt 16. The waist belt 16 provides a pair of coupling portions 18. The pair of coupling portions 18 of the waist belt 16 is positioned on the bag portion 12 of the backpack assembly 10 on the back or posterior side of the bearer. Each coupling portion 18 of the waist belt 16 provides a fastener 20 for fixing the coupling portions 18 together. In one exemplary embodiment, the fastener 20 is a combination of hooks and loops. The waist belt 16 encircling the waist of the bearer that will support the waist of the bearer.

In an exemplary embodiment of the invention, the bag portion 12 further includes a battery housing 22. The battery housing 22 is shaped to conform to a battery 52. One skilled in the art would understand the shape of the battery housing 22 may be any polygonal shape capable of housing the battery 52. In another exemplary embodiment, the battery housing 22 is a pocket permitting the battery 52 to slide into the battery housing 22.

In an exemplary embodiment of the invention, the battery housing 22 further includes a plurality of fasteners 24 extending across the battery housing 22 for coupling the battery 52 to the battery housing 22. In one exemplary embodiment, the plurality of fasteners 24 are provided as elastic braided straps for providing stability while allowing for easy removal for circuit adjustments or battery recharging. One skilled in the art would understand other fasteners may be implemented to stabilize the battery 52 within the battery housing 22. The battery housing 22 further provides coupling of the voltage booster 60 and circuit board 80.

In an exemplary embodiment of the invention, the bag portion 12 further includes a circuit board housing 30. The circuit board housing is shaped to conform to a circuit board. In one exemplary embodiment, the circuit board housing 30 may be rectangular shaped. One skilled in the art would understand other polygonal shapes for the circuit board housing may be utilized. The circuit board housing 30 is positioned adjacent the battery housing 22.

In an exemplary embodiment of the invention, the sleeve component 40 extends from a side of the bag portion 12. The sleeve component 40 features an E-shape. In another exemplary embodiment, the sleeve component 40 may be another polygonal shape permitting a limb to extend through. In another exemplary embodiment, the sleeve component 40 is non-fixed to the bag portion 12.

In an exemplary embodiment of the invention, sleeve component 40 further includes a thermistor housing 42. The thermistor housing 42 is positioned on the sleeve component 40 for coupling the thermistor 90 to the sleeve component 40. The thermistor housing 42 is a vinyl polysiloxane holder. One skilled in the art would understand the applicant's design is not the exclusive embodiment.

In an exemplary embodiment of the invention, the sleeve component 40 further includes a heater housing 44 positioned on the sleeve component 40 for coupling the heater 70 to the sleeve component 40.

In an exemplary embodiment of the invention, the sleeve component 40 further includes a contact area positioned on a surface that contacts the mammals skin.

The sleeve component 40 further includes a plurality of fastener 46 positioned at each end of the sleeve component 40.

In an exemplary embodiment of the invention, the electrical sub assembly 50 includes a battery 52. In one exemplary embodiment of the battery 52 the battery an A 3.7-volt, 1300 mAh lithium polymer battery. One of ordinary skill in the art would understand another battery may be used within the system.

In an exemplary embodiment of the invention, the battery 52 will provide enough power to heat the skin of the mammal to the desired temperature range based on energy absorption calculations. Additionally, the battery 52 is rechargeable.

In an exemplary embodiment of the invention, the electrical sub assembly 50 further includes in certain exemplary embodiments, a voltage booster 60. The voltage booster 60 ensures that the power supplied to a control system device matches its required input power. In one exemplary embodiment, the voltage booster 60 increased the battery 52 output of volts insuring the necessary voltage. The voltage booster 60 is coupled to the bag portion 12 by an adhesive. One skilled in the art would understand the applicant's design is not the exclusive embodiment.

In an exemplary embodiment of the invention, the electrical sub assembly 50 further includes a heater 70. The heater 70 may be a joule heater. In one exemplary embodiment, heater 70 may be 10 mm×10 mm rectangular heater, with a resistance of 44.6Ω, designed to operate with a current of 110 mA and a voltage of 5 volts. With these specifications, the heater 70 is sufficiently sized to effectively heat the area 5 mm proximal and 5 mm distal to the fractured site. One skilled in the art would understand alternative heaters may be utilized to cover a larger area of a chosen mammal site.

In an exemplary embodiment of the invention, the heater 70 converts electric energy into thermal energy. As the heater 70 has a reference temperature rise value of 30° C. from room temperature (approximately 22° C.), it efficiently raises the temperature of the mammal's bone within the desired range of 32° C.-42° C., facilitating the healing process.

In an exemplary embodiment of the invention, the electrical sub assembly 50 further includes a circuit board 80. The circuit board 80 controls the electrical sub assembly 50. The circuit board 80 includes a control system device 82. The control system device 82 may be the Arduino Nano 33 IoT. One skilled in the art would understand other designs may be implanted and act as the control system device 82. The circuit board 80 further includes a transistor in series with a 3-kΩ resistor and the heater 70 to increase the flow of current provided to the heater 70.

The control system device 82 is used as a microcontroller to control the heater 70 depending on the thermistor measurements. The control system device 82 has an operating voltage of 4.5 to 21 volts through the Vin pin.

In an exemplary embodiment of the invention, the circuit board 80 further includes a lead wire 84. The lead wire 84 may include a length of 210 mm which is extended from the circuit board 80 on the mammal's back down its arm or leg, spanning from the shoulder to the base of its hand or foot coupling to the heater 70. The safety feature built within the control system device 82 will shut the heater 70 off if the thermistor 90 reads a temperature higher than the specified temperature range.

In an exemplary embodiment of the invention, the electrical sub assembly 50 further includes a thermistor 90. The thermistor 90 is utilized for measuring the temperature of the mammal's skin surface. The thermistor 90 has a discrete negative temperature coefficient ambient temperature range of −40° C.-125° C., as well as a temperature accuracy range of 0° C.-70° C. with a temperature sensitivity of ±0.1° C., therefore it can accurately read temperature values within the range of 32° C.-42° C. It consists of a small epoxy-coated device with a maximum bead dimension of 2.4 mm and alloy lead wires for reduced thermal conductivity.

In an exemplary embodiment of the invention, the single thermistor 90 or a pair of thermistors 90 are connected to the circuit board 80 by wires making them the same length as the lead wire 84 extending to the heater 70. In order for the wires to travel down the arm or leg of the mammal and be sewn into the sleeve component 40. The thermistors 90 are secured in place using a vinyl polysiloxane material as the material has adequate thermal properties including the ability to withstand temperatures up to 93° C. preventing the heater 70 from melting the thermistor 90. One skilled in the art would understand other coupling material may be utilized.

In an exemplary embodiment of the invention, the at least two thermistors 90 were used in this design for safety precautions to avoid burning the mammal. If one out of the two thermistors 90 breaks, the second thermistor 90 is used as a precaution to ensure one of thermistors 90 is consistently reading accurate temperature values. However, in another exemplary embodiment, one thermistor 90 may be utilized within the assembly 1.

In an exemplary embodiment of the invention, the circuitry of the electrical sub assembly 50 is coated with a water resistant adhesive to mitigate water exposure. In another exemplary embodiment, the circuitry of the electrical sub assembly 50 is shrink-wrapped in order to mitigate water exposure.

In an exemplary embodiment of the invention, the voltage booster 60 is connected to the Vin pin to provide the circuit board 80 with power, indirectly from the battery 52. Each output pin on the control system device 80 emits a current of 7 mA. The heater 70 may require an operating current of 110 mA. Thus, pin D10 is used on the control system device 82 to connect a 3-kΩ resistor to a transistor and eventually the heater 70. One skilled in the art would understand the applicant's design is not the exclusive embodiment.

In an exemplary embodiment of the invention, the circuit board 80 is connected to the voltage booster 60 to boost the current to a safe amount for the heater 70. Pin D10 is utilized for the heater circuit to correlate with one of the pulse-width modulation pins on the microcontroller. Within the control system device 82 a code module controls the behavior of the microcontroller. The pulse width modulation is incorporated to turn the heater 70 on to its full power when the temperature measured by the thermistors 90 is less than the desired temperature. One degree of fluctuation is permitted, so the measured temperature will vary a maximum of ±1° C. from the set temperature. The desired temperature can be set by the user within the code module of the control system device 82. The control system device 82 uses integers, the temperature setpoint can be adjusted in one-degree increments, which falls below the specification for the temperature to be set in two-degree increments. However, when the temperature measured is above the desired temperature, the pulse width modulation is set to turn the heater 70 completely off. The thermistors 90 are connected to the control system device 82 at pins 2 and 5, which are analog pins. The code module that controls the control system device 82 calculates the temperature value based on the measured resistance of each of these thermistors 90 and displays the temperature measured in real-time.

A BLE Bluetooth capability of the control system device 82 is incorporated into the code that controls the microcontroller so the assembly 1 may be wireless and couple to a computing device. The computing device is a tablet computer or mobile phone which a user may utilize to monitor the assembly.

In an exemplary embodiment of the invention, the heater 70 will consistently heat a bone fracture in a mammal to a desired temperature setpoint input. The heater 70 will consist of a continuous heating system that can be attached to the arm or the leg of the mammal and will control the skin temperature of the bearer in two-degree (° C.) increments. The control system device 82 will actively monitor the temperature of the mammal's skin and a visual representation of the temperature will be displayed through the computing device.

It should be noted that while the exemplary embodiments and figures described herein illustrate the invention as applied to a specific mammal for testing purposes, one skilled in the art will appreciate that the invention is not limited to such an application. The bone osteogenesis assembly is designed to be adaptable for use with humans and larger mammals without departing from the scope or spirit of the invention. The components, including the wearable backpack assembly, sleeve component, and electrical sub-assembly, can be modified in size, configuration, and materials to accommodate anatomical and physiological variations across different species. This flexibility ensures the invention's applicability for both research and clinical use in promoting bone healing in diverse subjects, including but not limited to humans, dogs, and other large mammals.

While the present invention has been described primarily in connection with bone osteogenesis, it should be understood that, within the spirit and scope of this invention, it can also be applied to promote or enhance soft tissue healing. Various modifications and adaptations of the invention, as well as numerous embodiments and equivalents thereof, will be apparent to those skilled in the art, and all such variations are intended to be covered by the appended claims.

It should be noted that various changes and modifications to the embodiments described herein will be apparent to those skilled in the art. Such changes and modifications may be made without departing from the spirit and scope of the present invention and without diminishing its intended advantages. For example, various embodiments of the systems and methods may be provided based on various combinations of the features and functions from the subject matter provided herein.

1. A bone osteogenesis assembly, comprising:
a wearable backpack assembly including a bag portion,
the bag portion having a pair of shoulder straps, the bag portion further having:
a battery housing positioned on a rear of the bag portion;
a circuit board housing positioned adjacent the battery housing;
a sleeve component being a direct extension of the backpack assembly without an intervening structure, the sleeve component having a heater housing; and
an electrical sub assembly having a battery positioned within the battery housing, the electrical sub assembly further including a control system device positioned on a circuit board within the circuit board housing; the electrical sub assembly further including an internalized heating device housed within the heater housing, the internalized heating device is solely a joule heater, the internalized heating device coupled to the control system device and the battery, the heating device configured to continuously produce heat in twelve hour intervals in the sleeve component.

2. The bone osteogenesis assembly of claim 1, wherein the bag portion further includes a waist belt.

3. The bone osteogenesis assembly of claim 2, wherein the waist belt includes a pair of coupling portions.

4. The bone osteogenesis assembly of claim 1, wherein the battery housing further includes a plurality of fasteners for coupling to the battery.

5. The bone osteogenesis assembly of claim 1, wherein the sleeve component provides an E-shape.

6. The bone osteogenesis assembly of claim 1, wherein the electrical sub assembly further includes a thermistor.

7. The bone osteogenesis assembly of claim 6, wherein the sleeve component further includes a thermistor housing coupling the thermistor to the sleeve component.

8. The bone osteogenesis assembly of claim 7, wherein the sleeve component further includes a contact surface.

9. The bone osteogenesis assembly of claim 8, wherein the control system device includes a wireless coupling to a computing device.

10. The bone osteogenesis assembly of claim 9, wherein the battery is rechargeable.

11. The bone osteogenesis assembly of claim 10, wherein the control system device includes a code module to control a set determined temperature range.

12. The bone osteogenesis assembly of claim 11, wherein the control system device provides a safety feature to shut the internalized heating device off if the thermistor reads a temperature higher than the determined temperature range.

13. The bone osteogenesis assembly of claim 1, wherein the bag portion is composed of a polyester rayon spandex fabric mixture.

14. The bone osteogenesis assembly of claim 12, wherein a circuitry of the electrical sub assembly is coated with a water resistant material.

15. (canceled)

16. The bone osteogeneses assembly of claim 1, wherein the electrical sub assembly increases or decreases a flow of electric current to the internalized heating device providing a temperature increase or temperature decrease within the internalized heating device.

17. A continuous long term use bone osteogenesis healing assembly, comprising:
a wearable assembly having:
a battery housing positioned on a rear of the wearable assembly;
a circuit board housing positioned adjacent the battery housing;
a sleeve component being a direct extension of the wearable assembly, the sleeve component having solely an internal joule heating element, the internal joule heating element being the only heating element to heat consistently the sleeve component in continuous twelve-hour interval; and
a monitoring device positioned within the circuit board housing, the monitoring device maintaining an output of the internal joule heating element within a range between 32° C.-42° C. within the continuous twelve-hour interval.

18. A method of utilizing a long-term use bone osteogenesis healing assembly, comprising:
coupling a wearable assembly to a user;
securing a battery housing with a rechargeable battery on a rear of the wearable assembly;
securing a circuit board housing positioned adjacent the battery housing;
coupling a sleeve component protruding directly from the wearable assembly to a fracture site,
powering only an internal heating element, the internal heating element is solely a joule heater which couples to the rechargeable battery and fixing within the sleeve component and raising a temperature of the fracture site to a range between 32° C.-42° C. with continuously and solely produce heat in twelve hours intervals within the sleeve component; and
monitoring the continuously producing heat of the internal heating element through a control system device within the circuit board housing.
